(19) United States
(12) Patent Application Publication
(10) Pub. No.: US 2014/0017791 A1
(43) Pub. Date: Jan. 16, 2014

(54) Combining Biological Micro-Objects

(71) Applicant: Berkeley Lights, Inc., Emeryville, CA (US)

(72) Inventors: Kevin T. Chapman, Santa Monica, CA (US); Igor Y. Khandros, Orinda, CA (US); Gaetan L. Mathieu, Varennes (CA); Steven W. Short, Pleasanton, CA (US); Ming C. Wu, Moraga, CA (US)

(21) Appl. No.: 13/940,424

(22) Filed: Jul. 12, 2013

Related U.S. Application Data
(60) Provisional application No. 61/671,499, filed on Jul. 13, 2012.
(60) Provisional application No. 61/720,956, filed on Oct. 31, 2012.

(51) Int. Cl. C12N 13/00

(52) U.S. Cl. CPC C12N 13/00
USPC 435/450; 435/173.1; 435/283.1; 435/173.6

(57) ABSTRACT

Two or more biological micro-objects can be grouped in a liquid medium in a chamber. Grouping can comprise bringing into and holding in proximity or contact the micro-objects in a group, breaching the membrane of one or more of the micro-objects in a group, subjecting one or more of the micro-objects in a group to electroporation, and/or tethering to each other the micro-objects in a group. The micro-objects in the group can then be combined into a single biological object.

### CROSS REFERENCE TO RELATED APPLICATION(S)

This application is a non-provisional (and thus claims the benefit) of U.S. provisional patent application Ser. No. 61/671,499 (filed Jul. 13, 2012), which is incorporated by reference herein in its entirety. This application is also a non-provisional (and thus claims the benefit) of U.S. provisional patent application Ser. No. 61/720,956 (filed Oct. 31, 2012).

### BACKGROUND

In biological systems, it can be useful to combine multiple biological micro-objects. The present invention is directed to improved micro-fluidic devices and processes for selecting and grouping biological micro-objects and combining the grouped micro-objects into a combined biological object.

### SUMMARY

In some embodiments of the invention, a process of combining biological micro-objects can include selecting a first micro-object and a second micro-object from a plurality of micro-objects in a liquid medium in a micro-fluidic device. The process can further include grouping the first micro-object with the second micro-object in the liquid medium in the micro-fluidic device, and the process can also include, while the first micro-object and the second micro-object are grouped, combining the first micro-object and the second micro-object in the liquid medium to produce a combined object.

In some embodiments of the invention, an apparatus for combining biological micro-objects can include an enclosure, a grouping mechanism, and a combining mechanism. The enclosure can be for containing a liquid medium in which are disposed first micro-objects and second micro-objects. The grouping mechanism can be configured to group ones of the first micro-objects with ones of the second micro-object to produce micro-object groups such that each micro-object group includes one of the first micro-objects and one of the second micro-objects. The combining mechanism can be configured to combine the first micro-object and the second micro-object in each micro-object group.

### BRIEF DESCRIPTION OF THE DRAWINGS

FIG. 1A illustrates an example of a device for combing biological micro-objects of a first type with biological micro-objects of a second type according to some embodiments of the invention.

FIG. 1B is a side, cross-sectional view of the device of FIG. 1A.

FIG. 2 is a top, cross-sectional view of the device of FIG. 1A and illustrates operation of the device of FIG. 1A according to some embodiments of the invention.

FIG. 3 shows a cross-sectional partial side view of the device of FIG. 1A configured with an optoelectronic tweezers (OET) apparatus according to some embodiments of the invention.

FIG. 4 illustrates a partial, top, cross-sectional view of the device of FIG. 1A configured with the OET apparatus of FIG. 3 illustrating selecting and moving micro-objects with the OET apparatus of FIG. 3 according to some embodiments of the invention.

FIGS. 5A-5C illustrate an exampling of a combining mechanism for combining biological micro-objects from a first channel with biological micro-objects from a second channel according to some embodiments of the invention.

FIG. 6 shows an example of the combining region of the device of FIG. 1A that contains a combining chemical according to some embodiments of the invention.

FIG. 7 illustrates an example of the combining region of the device of FIG. 1A comprising spaced apart electrodes according to some embodiments of the invention.

FIG. 8A shows an example of the combining region of the device of FIG. 1A comprising opposing walls that define a compression passage according to some embodiments of the invention.

FIG. 8B illustrates an example of a breaching mechanism in the form of a knife with serrated blades according to some embodiments of the invention.

FIG. 9 illustrates a partial, top, cross-sectional view of the device of FIG. 1A configured with the OET apparatus of FIG. 3 illustrating a configuration of the device having virtual moving conveyors for picking up and moving micro-objects according to some embodiments of the invention.

FIG. 10A-10C illustrate another example of the device of FIG. 1A configured with the OET apparatus of FIG. 3 in which biological micro-objects are selected from and combined in different laminar flows in a chamber according to some embodiments of the invention.

FIG. 11A-11C illustrate yet another example of the device of FIG. 1A configured with the OET apparatus of FIG. 3 in which biological micro-objects are selected from flows in channels and combined at barriers in chambers between the channels according to some embodiments of the invention.

FIG. 12 illustrates operation of the device of FIG. 1A to combine more than two micro-objects according to some embodiments of the invention.

FIG. 13 shows an example process for combining biological micro-objects according to some embodiments of the invention.

### DETAILED DESCRIPTION OF EXEMPLARY EMBODIMENTS

This specification describes exemplary embodiments and applications of the invention. The invention, however, is not limited to these exemplary embodiments and applications or to the manner in which the exemplary embodiments and applications operate or are described herein. Moreover, the Figures may show simplified or partial views, and the dimensions of elements in the Figures may be exaggerated or otherwise not in proportion for clarity. In addition, as the terms “on,” “attached to,” or “coupled to” are used herein, one element (e.g., a material, a layer, a substrate, etc.) can be “on,” “attached to,” or “coupled to” another element regardless of whether the one element is directly on, attached, or coupled to the other element or there are one or more intervening elements between the one element and the other element. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

As used herein, “substantially” means sufficient to work for the intended purpose. The term “ones” means more than one.

The term “flow,” as used herein with reference to a liquid or gas, includes a continuous, pulsed, periodic, random, intermittent, or reciprocating flow of the liquid or gas.

As used herein, the term “biological micro-object” includes biological cells and compounds such as proteins, embryos, plasmids, oocytes, sperms, genetic material (e.g., DNA), transfection vectors, hydridomas, transfected cells, lipids, nanoparticles, and the like as well as combinations of the foregoing.

As used herein, “grouping” biological micro-objects means moving two or more biological micro-objects into contact or close proximity with each other. “Grouped” biological micro-objects are thus two or more biological micro-objects that are in contact or close proximity to each other.

The term “combine” when used in reference to grouped biological micro-objects encompasses fusing or transfecting the grouped biological micro-objects.

Fusing grouped biological micro-objects means combining the grouped biological micro-objects into a single combined micro-object.

Transfecting grouped biological micro-objects means injecting one or more of the grouped micro-objects as one or more transfection vectors into another of the grouped micro-objects.

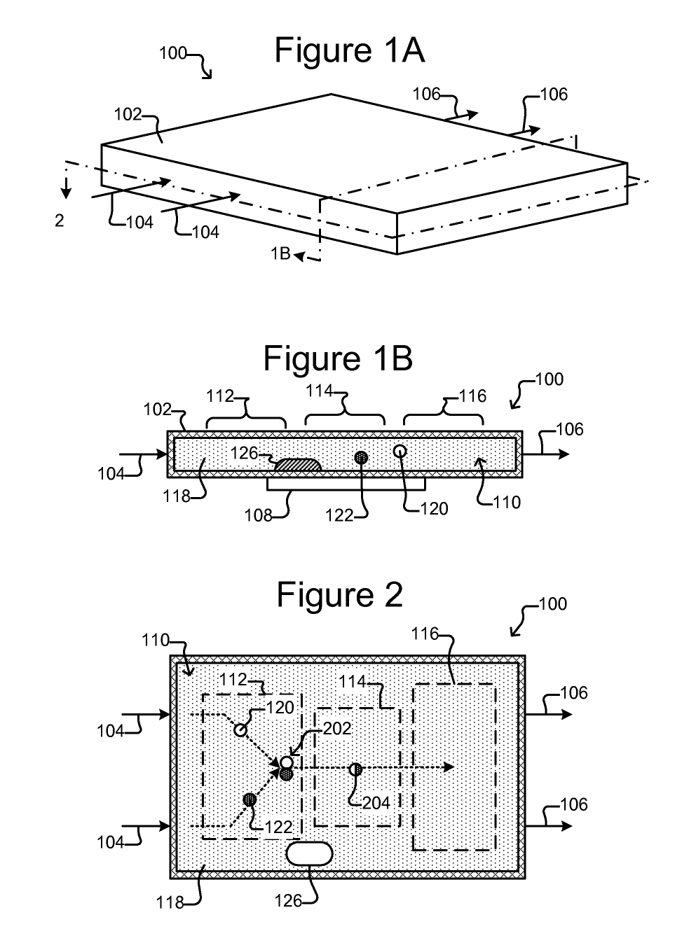

Embodiments of the invention can group biological micro-objects in a liquid medium in a chamber, and then combine (e.g., fuse) the grouped micro-objects into a combined (e.g., fused) biological micro-object. FIGS. 1A and 1B illustrate an example of a combining device 100 for grouping and combining (e.g., fusing) biological micro-objects according to some embodiments of the invention. As shown, the device 100 can comprise a housing 102 and a manipulation mechanism 108. In addition, some embodiments of the device 100 can comprise a breaching mechanism 126.

As shown in FIGS. 1A and 1B, the housing 102, can comprise one or more interior chambers 110 for holding a liquid medium 118 in which different types of biological micro-objects can be suspended. In the example shown in FIGS. 1A and 1B, a first type of biological micro-object 120 (hereinafter a first-type micro-object 120) and a second type of biological micro-object 122 (hereinafter a second-type micro-object 122) are suspended in the medium 118. (Hereinafter, the first-type micro-object 120 and the second-type micro-object 122 are also referred to collectively as micro-objects 120 and 122.) As shown in FIG. 1B, there can be a grouping region 112 and a combining region 114 in the chamber 110. As also shown, some embodiments can also include a sorting/selecting region 116.

The housing 102 can also comprise one or more inlets 104 through which the medium 118 and micro-objects 120 and 122 can be input into the chamber 110. An inlet 104 can be, for example, an input port, an opening, a valve, a channel, or the like. The device 100 can also comprise one or more outlets 106 through which the medium 118 with or without micro-objects 120 and 122 can be removed. An outlet 106 can be, for example, an output port, an opening, a valve, a channel, or the like.

The breaching mechanism 126 can be configured to breach (e.g., pierce) the membrane (e.g., the outer membrane) of one or more of the micro-objects 120, 122. For example, the breaching mechanism 126 can be a sharp physical object (e.g., a knife structure, a spear structure, or the like), which can be attached to the housing 102 and disposed inside the chamber 110. Another example of the breaching mechanism is a laser device configured to direct a laser beam at one or more of the micro-objects 120, 122. Yet another example of the breaching mechanism 126 is an ultrasonic device. Still another example of a breaching mechanism is an electrical stimulus device for applying an electrical stimulus to one or more of the micro-objects 120, 122.

The manipulation mechanism 108 can be configured to select and move individual micro-objects 120 and 122 in the chamber 110. For example, the manipulation mechanism can comprise a device for creating electrokinetic forces on the micro-objects 120 and 122 in the medium 118. For example, such devices (not shown) can include devices for creating dielectrophoresis (DEP) forces on selected ones of the micro-objects 120 or 122 to select and/or move the micro-objects. For example, the manipulation mechanism 108 can include one or more optical (e.g., laser) tweezers devices and/or one or more optoelectronic tweezers (OET) devices (e.g., as disclosed in U.S. Pat. No. 7,612,355, which is incorporated by reference herein). As yet another example, the manipulation mechanism 108 can include one or more devices (not shown) for moving a droplet of the medium 118 in which one or more of the micro-objects 120 and/or 122 are suspended. Such devices (not shown) can include electrowetting devices such as optoelectronic wetting (OEW) devices (e.g., as disclosed in U.S. Pat. No. 6,958,132, which is incorporated by reference herein).

FIG. 2 (which is a cross-sectional, top view of the device 100) illustrates operation of the device 100 according to some embodiments of the invention. As shown, a first-type micro-object 120 can be selected and grouped with a selected second-type micro-object 122 in the grouping region 112. (A set of grouped micro-objects 120 and 122 is labeled 202 in FIG. 2 and referred to herein as grouped micro-objects 202 or a group 202 of micro-objects.) Although two micro-objects 120, 122 are illustrated in a group 202, there can be more than two micro-objects 120, 122 in a group 202. Although not shown, there can be a plurality of first-type micro-objects 120 and second-type micro-objects 122 in the medium 118. The first-type micro-objects 120 and the second-type micro-objects 122 can be sorted based on one or more desired characteristics, and an individual first-type micro-object 120 can be selected based on such characteristics and grouped with an individual second-type micro-object 122 also selected for such characteristics. The foregoing sorting and selecting as well as grouping can be done in the grouping region 112.

If a breaching mechanism 126 is included in the device 100, the membrane of one or more of the micro-objects 120, 122 in a group 202 can be breached by the breaching mechanism 126. For example, if the breaching mechanism 126 is a sharp structure (e.g., a knife or spear structure), the group 202 can be moved into contact with the breaching mechanism 126 such that the sharp structure pierces the membrane of at least one of the micro-objects 120, 122 in the group 202. As another example, if the breaching mechanism 126 is a laser device, a laser beam can be directed at the group 202 to pierce the membrane of at least one of the micro-objects 120, 122 in the group 202. As yet another example, if the breaching mechanism 126 is an ultrasonic device, the ultrasonic device can be activated and the group 202 brought in sufficient proximity to the ultrasonic device to breach the membrane of at least one of the micro-objects 120, 122 in the group 202. As still another example, if the breaching mechanism 126 is an electrical stimulus device, the electrical stimulus device can be activated to apply an electrical stimulus to the group 202 to breach the membrane of at least one of the micro-objects 120, 122.

Before or after breaching the membrane of at least one of the micro-objects 120, 122 in the group 202, the grouped micro-objects 202 can be moved into the combining region 114, where the grouped micro-objects 202 can be subjected to one or more treatments (e.g., a chemical treatment, an electric field treatment, a pressure treatment, and/or the like) that combine the grouped micro-objects 202 into a combined micro-object 204. As also shown, the combined micro-object 204 can be moved into the sorting/selecting region 116 where the combined micro-object 204 can be sorted, tested, moved, stored, processed, output through an outlet 106, or the like.

In some embodiments, the micro-objects 120, 122 in the group 202 are not breached. Instead, the micro-objects 120, 122 can be tethered to each other, brought into and held in contact or close-proximity with each other, subjected to electroporation, or the like preparation for treatments that combine the grouped micro-objects 202 in the combining region 114. Moreover, the foregoing can be done after grouping in the grouping region 112 but before being subjected to combining treatment in the combining region 114.

Regardless, the first-type micro-objects 120 and the second-type micro-objects 122 can be different types of biological cells or compounds, and the combining of grouped micro-objects 202 can comprise fusing the two cells or compounds. For example, the first-type micro-objects 120 can be cells that produce a particular antibody (e.g., B-lymphocyte cells such as immunoglogulin (IgG)/antigen specific pre-plasmablast cells) (hereinafter referred to as anti-body-producing cells), and the second-type micro-object 122 can be cells that facilitate growth of the antibody-producing cells (e.g., immortalized myeloma cells) (hereinafter referred to as growth-facilitating cells). In such an example, the combining of a grouped set of an antibody-producing cell and a growth-facilitating cell (which can be an example of grouped micro-objects 202) can comprise fusing those cells to form a hydridoma (which can thus be an example of a combined micro-object 204). The hydridomas can then be grown and their secretion or expression of antibodies analyzed.

As another example, the first-type micro-objects 120 can be vectors to be injected by transfection into the second-type micro-objects 122, which can be biological cells. For example, the first-type micro-objects 120 can be liposomes carrying genetic material, and the second-type micro-objects 122 can be biological cells (e.g., procaryotic or eucaryotic cells) into which the genetic material is to be injected (e.g., by lipofection). In this example, the combining of a grouped set of a liposome and a biological cell (which can be an example of grouped micro-objects 202) can comprise injecting the liposome (carrying the genetic material) into the biological cell. The resulting combination of the biological cell with the injected liposome carrying the genetic material can be an example of the combined micro-object 204. The secretion or expression of materials such as protein by such transfected biological cells can then be monitored and analyzed.

As another example involving transfection, the first-type micro-objects 120 can be vectors comprising a gene knockdown material, and the second-type micro-objects 122 can be biological cells into which the gene knockdown material is to be injected by transfection. In this example, the combining of a grouped set of a vector carrying the gene knockdown material (e.g., small interfering ribonucleic acid (siRNA)) and a biological cell can comprise injecting the vector into the biological cell. The grouped biological cell and vector carrying the gene knockdown material can be an example of grouped micro-objects 202, and the resulting combination of the biological cell with the injected vector can be an example of the combined micro-object 204. The effect of the knockdown material on the biological cell can then be monitored and analyzed.

As yet another example, the first-type micro-objects 120 can be biological cells having one or more specific proteins and one or more common proteins expressed on a surface of the cell, and the second-type micro-objects 122 can be biological cells having only the common protein(s) but not the specific protein(s) expressed on a surface of the cell. The membrane of one or more of the micro-objects 120, 122 in each such group 202 can be breached as discussed above. Alternatively or in addition, the micro-objects 120, 122 in the group 202 can be tethered to each other by a tethering molecule, an antibody coated bead, or other tethering mechanism.

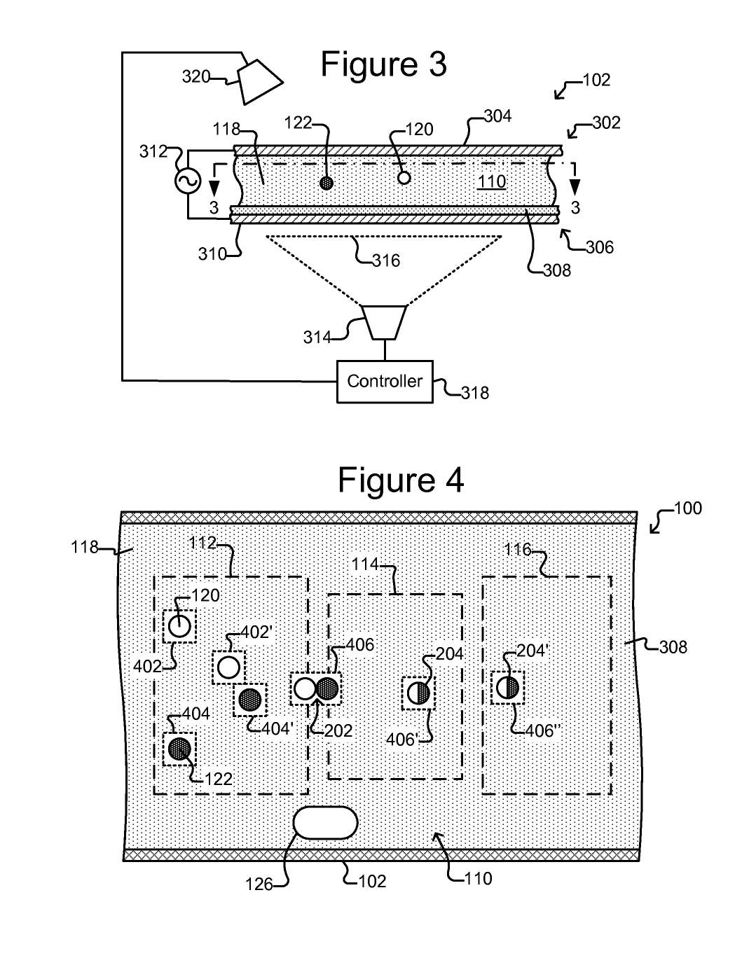

In some embodiments, the manipulation mechanism 108 can comprise an OET apparatus. FIG. 3 illustrates an example in which the manipulation mechanism 108 comprises an OET apparatus integrated into at least part of the housing 102. More specifically, FIG. 3 illustrates a side, cross-sectional view of a portion of the housing 102 of the device 100 in which at least a portion of an upper wall 302 of the housing 102 comprises an upper electrode 304, and at least a portion of a lower wall 306 comprises a photoconductive layer 308 and a lower electrode 310. As shown, the chamber 110 can be between the upper wall 302 and the lower wall 306.

As also shown, a biasing voltage 312 can be applied to the upper electrode 304 and the lower electrode 310. As is known, light projected onto an area of the photoconductive layer 308 can change the electric field between the upper electrode 304 and the lower electrode 310 in the vicinity of the illuminated area of the photoconductive layer 308. As is also known, depending on the frequency of the biasing voltage 312, this can attract or repel one or more of the micro-objects 120 and 122. A “virtual electrode” that attracts/repels a micro-object 120 or 122 can thus be created at any area or areas on the photoconductive layer 308 by illuminating the area or areas.

As shown in FIG. 3, the OET apparatus can comprise a light source 314 that can project any desired light pattern 316 onto the photoconductive layer 308 to selectively illuminate any area or areas of the photoconductive layer 308 and thus create virtual electrodes in any desired pattern on the photoconductive layer 308. The OET apparatus of FIG. 3 can also include an imaging device 320 (e.g., a camera or other vision device) to monitor the micro-objects 120 and 122 and a controller 320 for controlling the light source 314. The upper wall 302 and/or the lower wall 306 can be transparent.

The OET apparatus illustrated in FIG. 3 can be configured to cover one or more of the grouping region 112, the combining region 114, and/or the sorting/selecting region 116. Thus, the OET apparatus illustrated in FIG. 3 can be used to select and move micro-objects 120 and 122 in one or more of the grouping region 112, the combining region 114, and/or the sorting/selecting region 116.

The configuration of the OET apparatus shown in FIG. 3 is an example only, and variations are contemplated. For example, the light source 314 and the imaging device 320 can be in different locations than shown in FIG. 3. Thus, for example, the light source 314 and the imaging device 220 can be disposed on opposite sides of the housing 102 from what is shown in FIG. 3. As another example of a variation from what is shown in FIG. 3, the light source 314 and the imaging device 320 can be disposed on the same side of the housing 102, and a light refracting device (not shown) can refract light from the light source 314. As yet another example, the wall 306 can comprise a semiconductor in which are formed circuit elements such as phototransistors, photodiodes, transistors, or the like. As still another example, the electrode 304 can alternatively be part of the wall 106. In such an embodiment, the electrode 304 can be in contact with the medium 118 and electrically insulated from the electrode 310. The foregoing and other variations of the configuration shown in FIG. 3 are possible.

FIG. 4 illustrates an example in which the OET apparatus of FIG. 3 can be configured to cover the grouping region 112, the combining region 114, and the sorting/selecting region 116. As noted above, there can be a plurality of first-type micro-objects 120 and a plurality of second-type micro-objects 122 in the medium 118, which can be sorted and selected (e.g., in the grouping region 112) based on one or more characteristics. For example, as shown in FIG. 4 (which shows a cross-sectional, partial top view of the housing 102 of FIGS. 1A-2 configured as the OET apparatus of FIG. 3), one of the first-type micro-objects 120 can be selected by projecting a light pattern in the form of a light trap 402 (e.g., a light cage) from the light source 314 onto the photoconductive layer 308 around the first-type micro-object 120, which can trap the micro-object 120. A second-type micro-object 122 can similarly be selected by projecting a light trap 404 (e.g., a light cage) from the light source 314 onto the photoconductive layer 308 around the second-type micro-object 122, which can trap the micro-object 122. As shown in FIG. 4, this can be performed in the grouping region 112 of the chamber 110. The frequency of the biasing voltage 312 (see FIG. 3) can be such that the light traps 402, 404 repel the selected micro-objects 120 and 122. Alternatively, patterns of light can be created that attract a micro-object 120, 122.

The selected micro-objects 120 and 122 can then be moved within the grouping region 112 into proximity with each other by moving the light traps 402′ and 404′ on the photoconductive layer 308 as shown in FIG. 4. The light traps 402′ and 404′ can then be further moved on the photoconductive layer 308 to group the selected micro-objects 120 and 122 and thus form grouped micro-objects 202. The light traps 402′ and 404′ can be merged (e.g., replaced) with a light trap 406 projected from the light source 314 onto the photoconductive layer 308 around the grouped biological micro-objects 202. As shown in FIG. 4, the light trap 406 can be sized to maintain contact between the micro-objects 120 and 122 of the group 202. The grouped biological micro-objects 202 can be sorted (e.g., subjected to testing) to determine whether the grouping was successful, and grouped biological micro-objects 202 not meeting one or more criteria can be discarded.

The grouped micro-objects 202 can then be moved from the grouping region 112 to the combining region 114 by moving the light trap 406′ into the combining region 114 as shown. As mentioned, the grouped micro-objects 202 can be subject to one or more treatments in the combining region 114 that combine the grouped micro-objects 202 into a combined micro-object 204, which can then be moved into the sorting/selecting region 116. For example, the combined micro-object 204 can be moved from the combining region 114 into the sorting/selecting region 116 by moving the light trap 406″ into the sorting/selecting region 116 as shown. The light trap 406″ can then be turned off, releasing the combined micro-object 204 in the sorting/selecting region 116. As previously discussed, in the sorting/selecting region 116, the combined micro-objects 204 can be selected, sorted, tested, or otherwise processed or moved. For example, the combined micro-objects 204 can be sorted (e.g., by results of testing) by one or more characteristics, and combined micro-objects 204 not having such characteristics can be discarded.

Rather than turning the light trap 406″ off in the sorting/selecting region 116, the light trap 406″ can move the combined micro-object 204 in the sorting/selecting region 116 and thereby, for example, move the combined micro-objects 204 to particular locations or structures (e.g., a channel, an outlet 106, or the like) in or adjacent to the sorting/selecting region 116. For example, the combined micro-objects 204 can be moved to and held (e.g., in holding pens (not shown)) for a time period in the sorting/selecting region 116 or other location in the chamber 110. In such holding pens (not shown), the combined micro-objects 204 can be grown, cultured, give time to recover from the combining process, or the like.

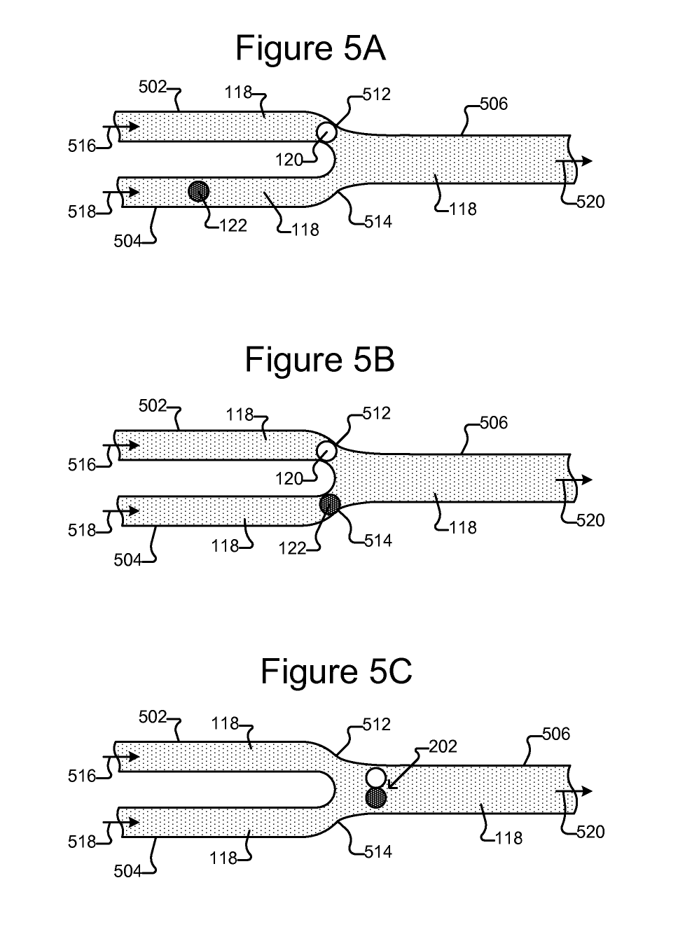

Because the OET apparatus of FIG. 3 can thus, among other things, select individual first-type micro-objects 120 and individual second-type micro-objects and group the selected first-type micro-object 120 with a selected second-type micro-object 122 to form a grouped micro-object 202, the OET apparatus of FIG. 3 (including any variation mentioned above) can be an example of a means for selecting a first micro-object 120 and a second micro-object 122, and the OET apparatus of FIG. 3 can also be an example of a means for grouping a first micro-object 120 and a second micro-object 122. FIGS. 5A-5C illustrate another example of a means for grouping a first micro-object 120 and a second micro-object 122.

FIGS. 5A-5C show a first channel 502 and a second channel 504 connected by passages 512, 514 to a third channel 506. For example, although not shown in FIGS. 1A, 1B, and 2, housing 102 can comprise the channels 502, 504, 506. For example, inlets 104 can be inputs to the first and second channels 502, 504; the channels 502, 504, 506 can comprise the combining region 112; and an output of the third channel 506 can be disposed in the combining region 114. (See FIGS. 1A, 1C, and 2.)

In operation, one or more of the first-type micro-objects 120 can be provided in a flow 516 of the medium 118 in the first channel 502, and one or more of the second-type micro-objects 122 can be provided in a flow 518 of the medium 118 in the second channel 504. The width of the first channel 502 can be greater than the size of the first-type micro-objects 120 so that the first-type micro-objects 120 readily move with the flow 516 in the first channel 502 to the first passage 512, which connects the first channel 502 to the third channel 506. Similarly, the width of the second channel 504 can be greater than the size of the second-type micro-objects 122 so that the second-type micro-objects 122 readily move with the flow 518 in the second channel 504 to the second passage 514, which connects the second channel 504 to the third channel 506.

The width of the first passage 512, however, can be sufficiently smaller than the first-type micro-objects 120 such that friction forces cause a first-type micro-object 120 to stop in the first passage 512 as shown in FIG. 5A. The width of the second passage 514 can likewise be sufficiently smaller than the second-type micro-objects 122 such that friction forces cause a second-type micro-object 122 to stop in the passage 514 as shown in FIG. 5B.

The widths of the first and second passages 512, 514 can also be sized such that the combined pressure of the flows 516, 518 in the channels 502, 504 can become sufficient to overcome the foregoing friction forces while both a first-type micro-object 120 is stopped and held in the first passage 512 and a second-type micro-object 122 is stopped and held in the second passage 514 as illustrated in FIG. 5B. As shown in FIG. 5C, this can cause the micro-objects 120, 122 to move from the passages 512, 514 into the third passage 506 as a group 202 of the micro-objects. The now grouped 202 micro-objects can then move with the flow 520 of medium 118 in the third channel 506. For example, as discussed above, the device 100 of FIGS. 1A, 1B, and 2 can be configured with the channels 502, 504, 506 of FIG. 5 such that the flow 520 in the third channel 506 moves the grouped 202 micro-objects into the combining region 114.

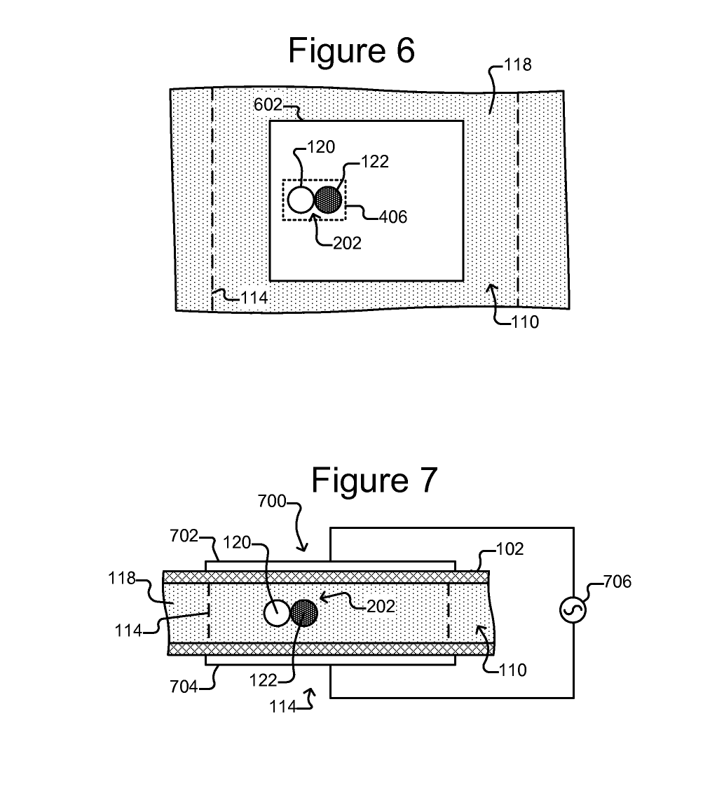

FIGS. 6-8A illustrate examples of configurations of the combining region 114 for performing various treatments to combine grouped micro-objects 202 into a combined micro-object 204 according to some embodiments of the invention.

As shown in FIG. 6, in some embodiments, the combining region 114 can comprise a chemical 602 for performing a chemical treatment of the grouped micro-objects 202. The chemical 602 can effect combining the grouped micro-objects 202. For example, the chemical 602 can effect fusing the grouped micro-objects 202, for example, where the micro-objects 120 and 122 of the group 202 are two different cell types. The chemical 602 can be disposed in a channel, chamber, or the like (not shown) in the combining region 114, and the grouped micro-objects 202 can be moved into the chemical 602 for a sufficient time to effect combing the grouped micro-objects 202 into the combined micro-object 204. In some embodiments, the combining chemical can be polyethylene glycol (PEG), the Sendai virus, or the like. The grouped micro-objects 202 can be moved into and within the chemical 602, for example, by moving the light trap 406 generally as illustrated in FIG. 6 or in a fluidic flow. A channel, chamber, or the like for holding a combining chemical is thus an example of a combining means.

FIG. 7 illustrates another example configuration of the combining region 114 according to some embodiments of the invention. As shown, the combining region 114 can comprise an electric field treatment mechanism 700, which can comprise a first electrode 702 and a second electrode 704 to which a biasing voltage 706 (e.g., a direct current (DC) or alternating current (AC) voltage) is applied. The type (DC or AC), voltage level, and frequency (if AC) of the biasing voltage 706 can be selected to effect combining a set of grouped micro-objects 202. Grouped micro-objects 202 can be moved between the electrodes 702 and 704 for a sufficient time to effect combining the grouped micro-objects 202 into a combined micro-object 204. The grouped micro-objects 202 can be moved into and within the electric field treatment mechanism 700, for example, by moving the light trap 406 generally as illustrated in FIG. 7 or in a fluidic flow. The opposing electrodes 702, 704 are thus another example of a combining means.

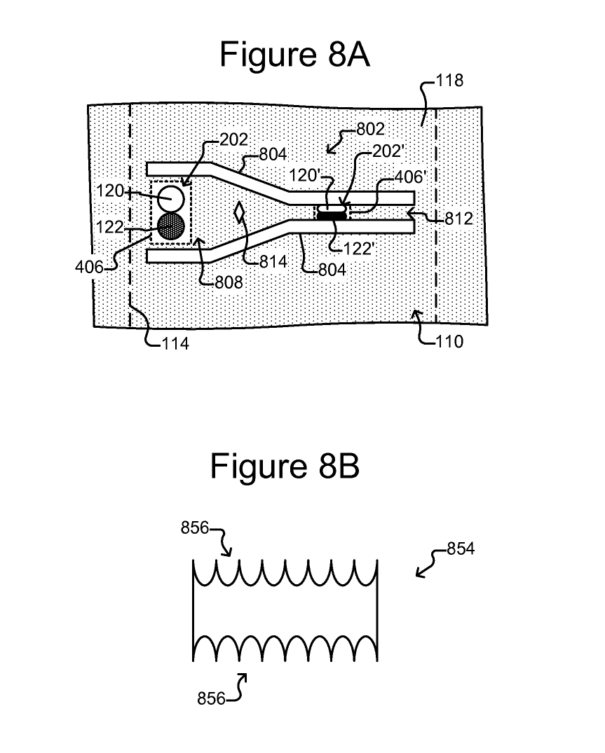

FIG. 8A illustrates yet another example configuration of the combining region 114 according to some embodiments of the invention. As shown, the combining region 114 can comprise a compression mechanism 802, which can comprise generally opposing walls 804. The opposing walls 804 can taper from relatively widely spaced, where the walls 804 define an entry space 808, to relatively narrowly spaced, where the walls 804 define a compression passage 812. The entry space 808 can be sufficiently wide to receive grouped micro-objects 202, and the compression passage 812 can be narrow enough to apply sufficient pressure to the grouped micro-objects 202 to combine the grouped micro-objects 202 to produce a combined micro-object 204. For example, a width of the compression passage 812 can be less than the sum of the sizes of the first micro-object 120 and the second micro-object 122.

The grouped micro-objects 202 can be moved into the entry space 808 and then through the compression passage 812 as shown in FIG. 8A. Pressure on the grouped micro-objects 202 from the compression passage 812 can effect combining the grouped micro-objects 202. The grouped micro-objects 202 can be moved, for example, by moving the light trap 406 generally as illustrated in FIG. 8A or in a fluidic flow. Opposing walls forming a compression passage 812 are thus another example of a combining means.

FIG. 8A also illustrates an example of a breaching mechanism 814 in the form of a knife-like or spear-like structure for breaching the membrane of one or more of the micro-objects 120, 122 of the group 202. The group 202 can be moved such that at least one of the micro-objects 120, 122 make sufficient contact with the breaching mechanism 814 to pierce the membrane of one or more of the micro-objects 120, 122 in the group 202. The group 202 can be moved into contact with the breaching mechanism 814 by moving the light trap 406 or in a fluidic flow. As noted above, the membranes of the micro-objects 120, 122 need not be breached, and thus, some embodiments of the compression mechanism 802 do not include the breaching mechanism 814.

As mentioned, the breaching mechanism 814 can be in the form of a knife-like structure, which can comprise one or more blades for breaching the membrane of one or more of the micro-objects 120, 122. Such blades can be smooth blades. Alternatively, the blades of the breaching mechanism 814 can be serrated. FIG. 8B illustrates an example of a breaching mechanism in the form of a knife 854 comprising serrated blades (edges) 856. The knife 854 can replace the breaching mechanism 814 in FIG. 8A or any other breaching mechanism illustrated in the figures or mentioned herein. The knife 854, as well as some other embodiments of the breaching mechanism 126, 814 can be, for example, a distinct structure or etched into or from the housing 102. For example, the housing 102 can comprise an etchable material such as silicon, and the knife 854 can be etched into or from the silicon using, for example, deep reactive ion etching or the like.

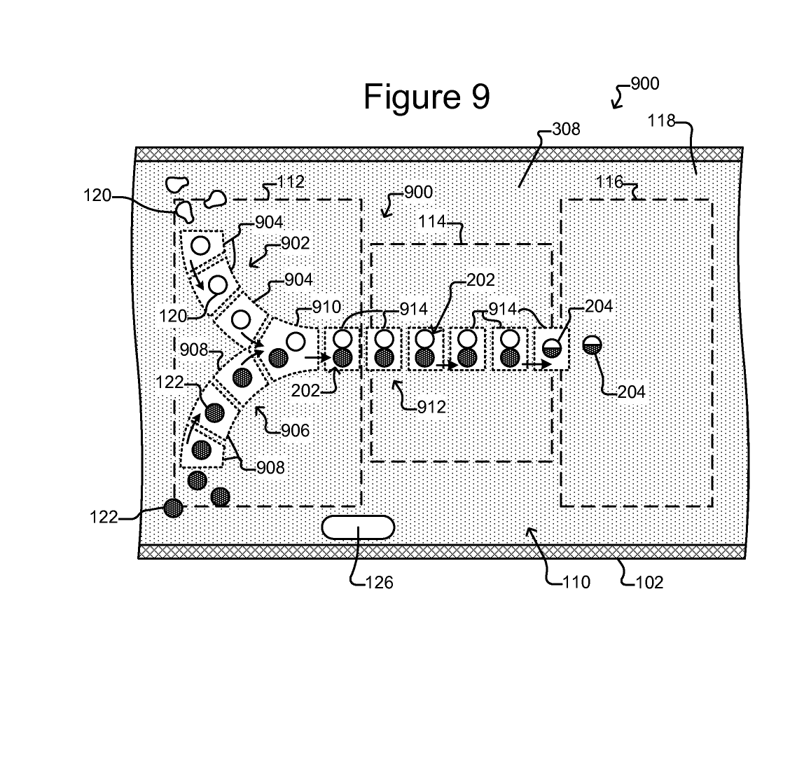

FIGS. 9-11C illustrate additional examples of micro-fluidic devices 900, 1000, 1100 according to some embodiments of the invention. Each of the devices 900, 1000, 1100 illustrate an example of a specific configuration of the device 100 discussed above.

As shown, the device 900 of FIG. 9 (which shows a cross-sectional, partial top view of the housing 102 of FIGS. 1A and 1B configured with the OET apparatus of FIG. 3) can be configured with a virtual conveyor system device 900. The OET apparatus of FIG. 3 (including any variation discussed above) can be configured to project the light pattern 316 on the photoconductive layer 308 in the form of a virtual moving conveyor system device 900, which can comprise virtual moving conveyors 902, 906, 912. As shown, each conveyor 902, 906, 912 can comprise moving light traps 904, 908, 910, 914. For example, a first moving conveyor 902 can comprise a series of moving light traps 904, and a second moving conveyor 906 can comprise a series of moving light traps 908. A third moving conveyor 912 can comprise a series of moving light traps that include an initial combined light trap 910 and additional light traps 914.

As shown, the moving light traps 904 of the first conveyor 902 can pick up (an example of selecting) individual first-type micro-objects 120 and move those individual first-type micro-objects 120 to the combined light trap 910 of the third conveyor 912. Similarly, the moving light traps 908 of the second conveyor 906 can pick up (an example of selecting) individual second-type micro-objects 122 and move those individual second-type micro-objects 122 to the combined light trap 910. This can result in a first-type micro-object 120 and a second-type micro-object 122 being brought together in the first combined light trap 910 as shown. As the first combined light trap 910 moves in the series of light traps 910 and 914 of the third conveyor 912, the size of the additional traps 914 can be adjusted as needed to bring the micro-objects 120 and 122 into contact and thus form grouped micro-objects 202 in a light trap 914. As shown, the third conveyer 912 can move grouped micro-objects 202 in each light trap 914 through the combining region 114. The breaching mechanism 126 can breach the membrane of one or more of the micro-objects in each group 202 before or after the third conveyer 912 moves the group 202 into the combining region 114. Although not shown, the micro-objects 120, 122 can be sorted in the grouping region 112 generally as discussed above.

As discussed above, the grouped micro-objects 202 can be subjected to one or more treatments in the combining region 114 that combine grouped micro-objects 202 into a combined micro-object 204. Examples of such treatments include any mentioned above including the examples of such treatments illustrated in FIGS. 6-8A. Thus, for example, the combining region 114 in FIG. 9 can include one or more of the combining chemical 602 of FIG. 6, the electric field treatment mechanism 700 of FIG. 7, the compression mechanism 802 of FIG. 8A, or any combination of the foregoing treatments. In the foregoing example, the third conveyer 912 can move each set of grouped micro-objects 202 in a light trap 914 through the combining chemical 602 of FIG. 6, the electric field treatment mechanism 700 of FIG. 7, and/or the compression mechanism 802 of FIG. 8A.

As shown in FIG. 9, the third conveyor 912 can move the resulting combined micro-objects 204 into the sorting/selecting region 116. As also shown, the third conveyor 912 can release the combined micro-object 204 in the sorting/selecting region 116. As previously discussed, in the sorting/selecting region 116, the combined micro-objects 204 can be selected, sorted, or otherwise processed or moved. Alternatively, the third conveyor 912 can extend farther into the sorting/selecting region 116 and thereby, for example, convey the combined micro-objects 204 to particular locations or structures (e.g., a channel, an outlet 106, or the like) in or adjacent to the sorting/selecting region 116.

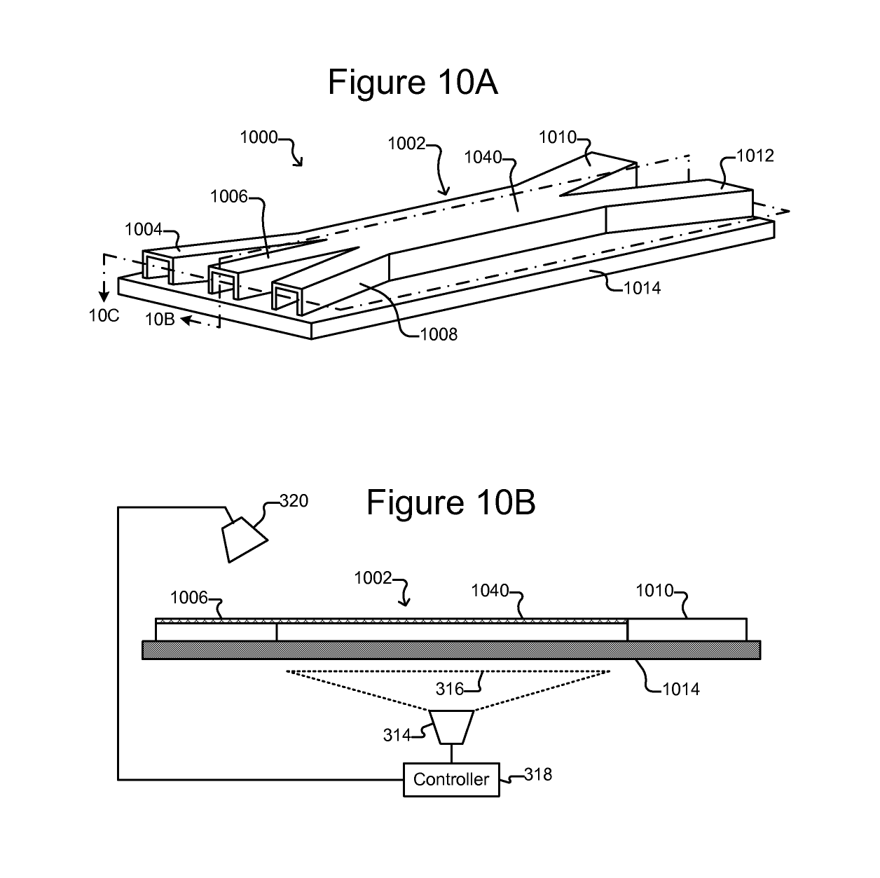

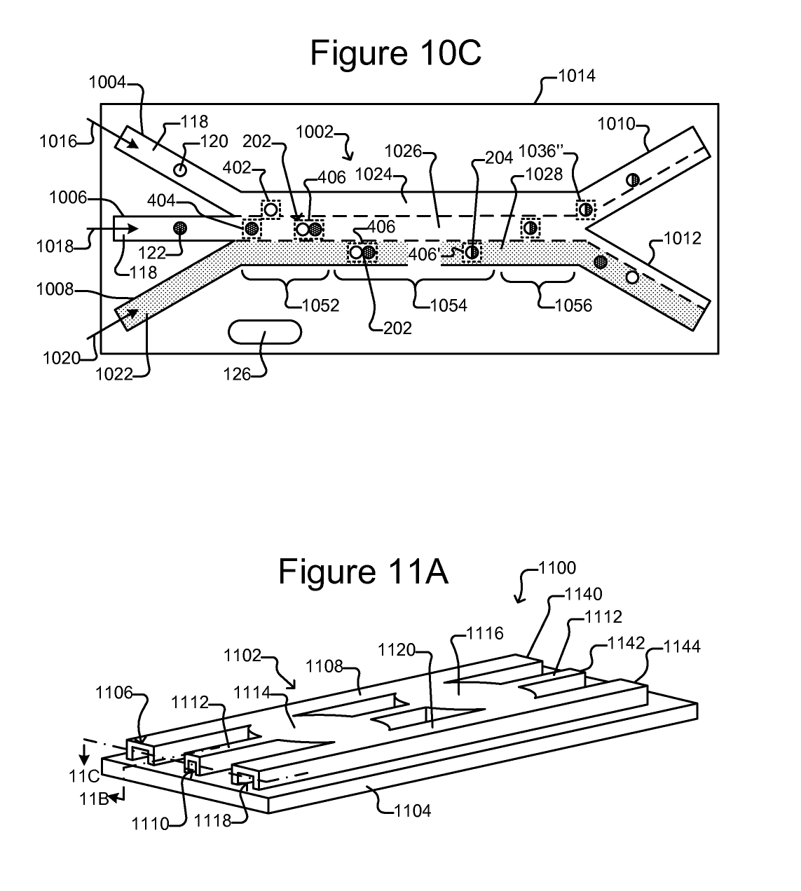

Referring now to the device 1000 of FIGS. 10A-10C, that device 1000 can comprise a base 1014 on which inlet channels 1004, 1006, and 1008, a chamber 1002, and outlet channels 1010 and 1012 can be disposed. Inputs to the inlet channels 1004, 1006, and 1008 can be examples of the inlets 104 in FIGS. 1A and 1B, and the outputs from the outlet channels 1010 and 1012 can be examples of the outlets 106 in FIGS. 1A and 1B. The channels 1004, 1006, 1008, 1010, 1012 and the chamber 1002 can similarly be an example of the housing 102 in FIGS. 1A and 1B. There can be, of course, more or fewer of the inlet channels 1004, 1006, and 1008 and/or outlet channels 1010 and 1012.

Although not shown, an upper wall 1040 of the chamber 1002 can be configured like the upper wall 302 in FIG. 3, and at least a portion of the base 1014 that corresponds to the chamber 1002 can be configured like the lower wall 306 in FIG. 3 (including any variation discussed above). As shown, the device 1000 can include the light source 314 of FIG. 3 for projecting patterns of light 316 onto at least a portion of the base 1014 that corresponds to the chamber 1002. Generally in accordance with the discussion above with respect to FIGS. 3 and 4, light traps 402 and 406 (see FIG. 10C) can be selectively created to select, move, and/or group micro-objects 120 and 122 in the chamber 1002. These light traps 402 and 406 can be the same as the light traps 402, 404, and 406 discussed above with respect to FIG. 4.

FIG. 10C (which is a cross-sectional, top view of the device 1000) illustrates operation of the device 1000 according to some embodiments of the invention. As shown, a flow 1016 of the liquid medium 118 in which first-type micro-objects 120 are suspended can be input into the inlet channel 1004. This can create a laminar flow 1024 of the liquid medium 118 in the chamber 1002 from the inlet channel 1004 to the outlet channel 1010. Similarly, a flow 1018 of the liquid medium 118 in which second-type micro-objects 122 are suspended can be input into the inlet channel 1006, which can create a laminar flow 1026 of the liquid medium 118 in the chamber 1002 from the inlet channel 1006 to the outlet channels 1010 and 1012 as shown. A flow 1020 of a combining chemical 1022 (which can be the same as or similar to the combining chemical 602 discussed above with respect to FIG. 6) can be input into the inlet channel 1008, which can create a laminar flow 1028 of the combining chemical 1022 in the chamber 1002 from the inlet channel 1008 to the outlet channel 1012.

As shown in FIG. 10C, the flow 1016 of the medium 118 in the inlet channel 1004 can move first-type micro-objects 120 into the chamber 1002, and the flow 1018 of the medium 118 in the inlet channel 1006 can also move second-type micro-objects 122 into the chamber 1002. As also shown, one of the first-type micro-objects 120 can be selected in the laminar flow 1024 by projecting a light pattern from the light source 314 (see FIG. 3) in the form of a light trap 402 around the first-type micro-object 120, and one of the second-type micro-objects 122 can be selected in the laminar flow 1026 by projecting a light pattern from the light source 314 in the form of a light trap 404 around the second-type micro-object 122.

The selected micro-objects 120 and 122 can then be moved into contact and the light traps 402, 404 merged (as discussed above with respect to FIG. 4) to form a light trap 406 around the now grouped micro-objects 202. The light trap 406 can be sized to maintain contact between the micro-objects 120 and 122 of the group 202. Generally as discussed above, the breaching mechanism 126 can breach the membrane of one or more of the micro-objects in each group 202.

The grouped micro-objects 202 can be moved by moving the light trap 406 into the laminar flow 1028 of the combining chemical 1022 as shown. The grouped micro-objects 202 can be in the combining chemical 1022 for a time period sufficient for the chemical 1022 to combine the grouped micro-objects 202 and thus create a combined micro-object 204 generally as discussed above.

Still referring to FIG. 10C, the combined micro-object 204 can then be moved out of the laminar flow 1028 of the combining chemical 1022 and then sorted. For example, it can be determined whether the grouped micro-objects 202 successfully combined into a combined micro-object 204. Those that successfully combined can be moved into the outlet channel 1010, which can be an output for successfully combined micro-objects 204. The micro-objects 120 and 122 of grouped micro-objects 202 that did not successfully combine can be moved into the outlet channel 1012, which can be an output for waste.

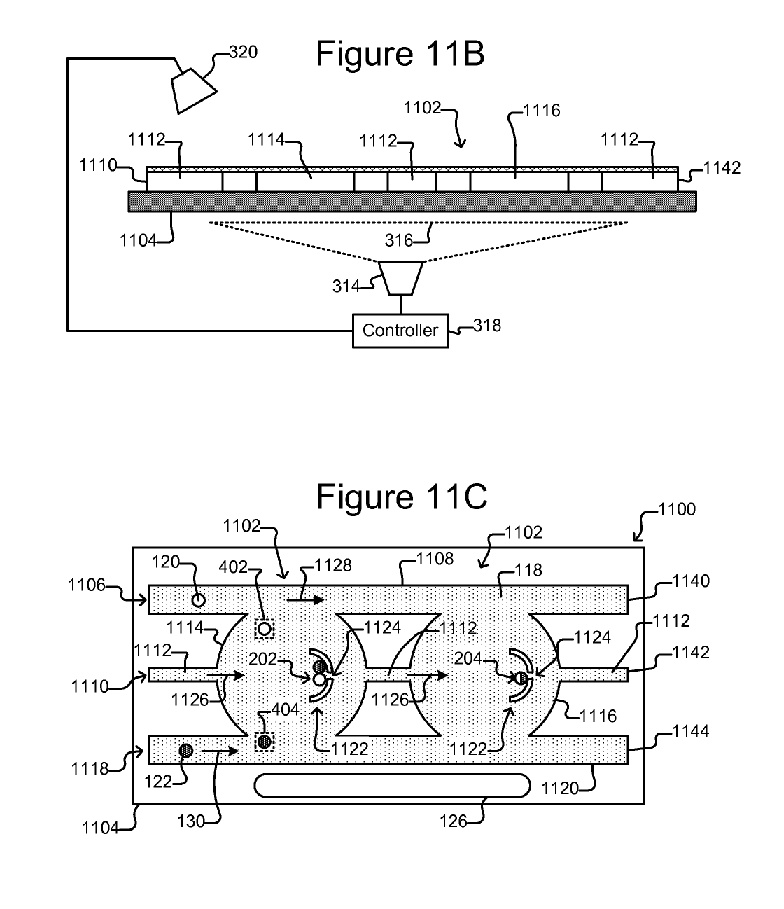

Referring now to FIGS. 11A-11C, the device 1100 can comprise a base 1104 on which a housing 1102 (which can be an example of the housing 102 of FIGS. 1A-2) is disposed. As also shown, the housing 1102 can comprise one or more channels 1108 and 1120 (two are shown but there can be fewer or more) and a flow channel 1112 (one is shown but there can be more). The channels 1108, 1112, and 1120 can lead to one or more chambers 1114 and 1116 (two are shown but there can be fewer or more). Inputs 1106, 1110, and 1118 of the channels 1108, 1112, and 1120 can be examples of the inlets 104, and outputs 1140, 1142, and 1144 of the channels can be examples of the outlets 106 in FIGS. 1A-2.

Although not shown, an upper wall of the housing 1102 can be configured like the upper wall 302 in FIG. 3, and at least a portion of the base 1104 can be configured like the lower wall 306 in FIG. 3 (including any variation of the apparatus shown in FIG. 3). As shown in FIG. 11B, the device 1100 can also include the light source 314 of FIG. 3 for projecting patterns of light 316 onto the base 1104.

Generally in accordance with the discussion above with respect to FIGS. 3 and 4, as illustrated in FIG. 11C (which is a cross-sectional, top view of the device 1100), light traps 402 can be selectively created to select first-type micro-objects 120 from a flow 1128 of the medium 118 in the first channel 1108 and move the selected micro-object 120 to a barrier 1122 in a chamber 1114, 1116 disposed between the channels 1108, 1120. Similarly, light traps 404 can be selectively created to select second-type micro-objects 122 from a flow 1130 of the medium 118 in the second channel 1120 and move the selected micro-object 122 to the barrier 1122. Groups 202 of the micro-objects 120, 122 can thus be formed at the barriers 1122. In FIG. 11C, an example of a group 202 of micro-objects 120, 122 is shown at the barrier 1122 in chamber 1114.

The breaching mechanism 126 can, as discussed above, breach the membrane of one or more of the micro-objects 120, 122 in a group 202. The barriers 1122 can be physical, virtual, or a combination of physical and virtual.

A flow 1126 of a chemical (e.g., like chemical 602 or 1022) can be provided in the channel 1110. Openings 1124 in the barriers 1122 can allow the flow 1126 to flow through the barriers 1122. As a result, the micro-objects 120, 122 in each group 202 can combine into a combined micro-object 204 as shown, for example, at the barrier 1122 in the chamber 1116 in FIG. 11C. Combined micro-objects 204 can be selected and moved (e.g., with light traps like traps 402, 404) out of the chambers 1114, 1116.

As noted above, the membranes of grouped 202 micro-objects 120, 122 need not be breached, and thus, some embodiments of the devices 900, 1000, 1100 in FIGS. 9A-11C do not include the breaching mechanism 126. As also noted above, grouped 202 micro-objects 120, 122 can instead be subjected to electroporation, tethered together, held in contact or close proximity to each other, or the like.

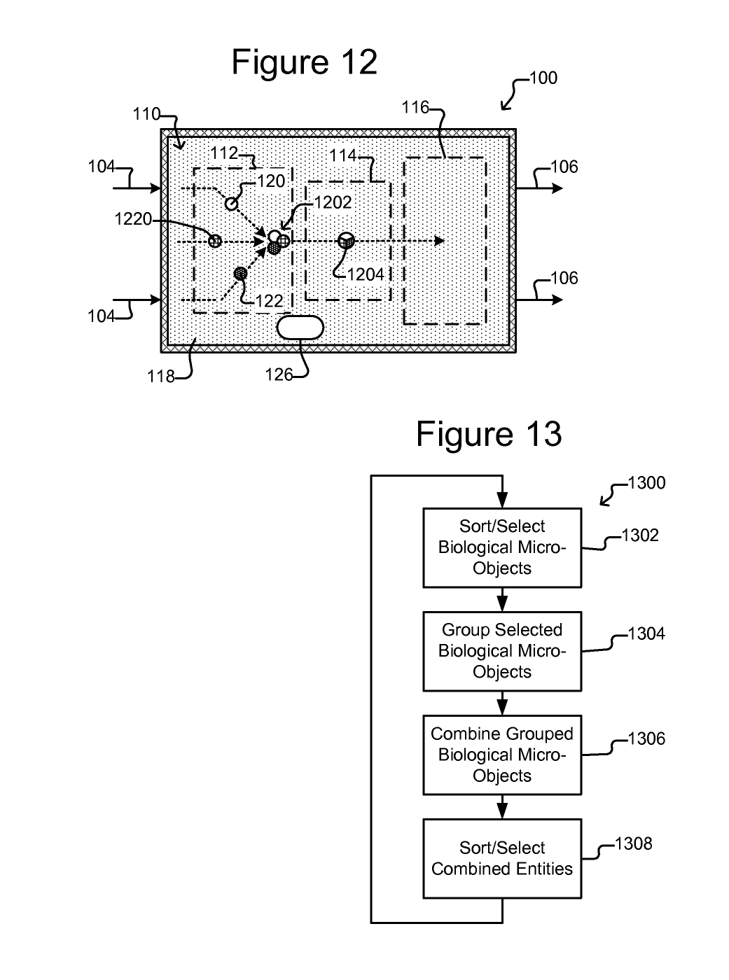

The devices 100, 900, 1000, 1100 illustrated in the figures and described herein are examples only, and variations are contemplated. For example, although two micro-objects 120, 122 are illustrate as being grouped and combined in the examples illustrated in the figures and discussed herein, more than two micro-objects can be grouped and combined. FIG. 12 illustrates an example of operation of the device 100 (see, e.g., FIGS. 1A, 1B, and 2) in which a plurality of micro-objects 120, 122, 1220 (three are shown but there can be more) are grouped in the grouping region 112 and combined in the combining region 116. The micro-objects 1220 can enter the device 100 through one of the inlet ports 104 or in some other manner. As noted above, the device 100 can have more than two inlet ports 104.

FIG. 12 illustrates operation of the device 100 as shown in FIG. 2 except a plurality of micro-objects 120, 122, 1220 are grouped in the grouping region 112. The grouping of the micro-objects 120, 122, 1220 can be as described above with respect to FIG. 2 except more than two micro-objects 120, 122, 1220 are selected and grouped to form grouped 1202 micro-objects. Each group 1202 can thus comprise three or more micro-objects 120, 122, 1220. The micro-objects 120, 122, 1220 can be selected and combined into groups 1202 in any manner described herein for selecting and combining micro-objects 120, 122 into groups 202. In the combining region 114, each grouped 1202 micro-objects 120, 122, 1220 are combined into a combined micro-object 1204. Each group 1202 of micro-objects 120, 122, 1220 can be combined in any manner described herein for combining a group 202 into a combined micro-object 204.

The micro-object 1220 can be any of the types of micro-objects discussed above with regard to micro-objects 120, 122. Moreover, micro-object 1220 can be the same as or different than either of the micro-objects 120, 122. For example, the micro-object 120 can be a biological cell, and the micro-objects 122, 1220 can be transfection vectors such as plasmids (or the like) having selectable markers. As just one such example, the micro-object 120 can be a cell such as a Chinese hamster ovary (CHO) cell, the micro-object 122 can be a specific antibody heavy chain within a lipid nano-particle (or the like), and the micro-object 1220 can be a specific antibody light chain within a lipid nano-particle (or the like).

As mentioned, although three micro-objects 120, 122, 1220 are illustrated in FIG. 12 being grouped into group 1202, and combined into a combined micro-object 1204, more than three such micro-objects can be grouped into group 1202, and combined into a combined micro-object 1204. Moreover, any of the devices 900, 1000, 1100 illustrated and discussed herein can group and combine more than two micro-objects 120, 122 generally as illustrated in FIG. 12 and discussed above.

FIG. 13 shows an example of a process 1300 for combining biological micro-objects according to some embodiments of the invention. At step 1302, micro-objects can be sorted and individual micro-objects selected. For example, generally as discussed above, the medium 118 in any of the devices discussed above (e.g., devices 100, 900, 1000, 1100) can comprise a plurality of first-type micro-objects 120 and a plurality of second-type micro-objects 122, which can be sorted by one or more characteristics. Individual ones of the first-type micro-object 120 and the second-type micro-object 122 having a desired characteristic or that meet a particular criterion can be selected at step 1302. As noted above, there can be more than two micro-object types 120, 122. For example, as illustrated in FIG. 12, there can be three or more types of micro-objects 120, 122, 1220.

At step 1304, individual ones of the biological micro-objects selected at step 1302 can be grouped. For example, as illustrated in FIG. 2, a selected first-type biological micro-object 120 (see FIGS. 1A-2) can be grouped with a selected second-type biological micro-object 122 to form a group 202. As another example, as shown in FIG. 12, the selected micro-objects 120, 122 can be grouped with a third-type micro-object 1220. The foregoing, and thus step 1304, can be accomplished in any manner illustrated in the drawings or discussed above for creating a group 202, 1202 of micro-objects 120, 122, 1220.

Generally in accordance with discussions above, step 1304 can include breaching the membrane of (e.g., by any mechanism discussed above) or subjecting to electroporation one or both of the grouped 202, 1202 micro-objects 120, 122, 1220. Alternatively or in addition, step 1304 can include bringing and holding the micro-objects 120, 122, 1220 in a group 202, 1202 into contact or close proximity. Step 1304 can also include tethering the micro-objects 120, 122, 1220 in a group 202, 1202 to each other. Also generally in accordance with discussions above, step 1304 can include testing and sorting the grouped 202, 1202 micro-objects 120, 122, 1220 and selecting the grouped 202, 1202 micro-objects 120, 122, 1220 that have one or more characteristics or that meet one or more criteria.

At step 1306, the micro-objects 120, 122, 1220 in the group 202, 1202 created at step 1304 can be combined (e.g., fused) into a combined micro-object 204, 1204 which can be accomplished in any manner illustrated in the drawings or discussed above. Generally as noted above, a combined micro-object 204, 1204 created at step 1306 can be held in a holding pens (not shown in the drawings) in any of the devices 100, 900, 1000, 1100 illustrated and discussed herein. For example, a combined micro-object 204, 1204 can be cultured or provided a recovery period in such holding pens.

As shown, steps 1302-1306 can be repeated one or more times to produce a plurality of combined micro-objects 204, 1204. At step 1308, the combined micro-objects 204, 1204 can be tested, sorted, and/or selected and sorted in any manner illustrated in the drawings or discussed above.

Although specific embodiments and applications of the invention have been described in this specification, these embodiments and applications are exemplary only, and many variations are possible.

We claim:

1. A process of combining biological micro-objects, said process comprising:
selecting a first biological micro-object and a second biological micro-object from a plurality of micro-objects in a liquid medium in a micro-fluidic device;
grouping said first micro-object with said second micro-object in said liquid medium in said micro-fluidic device; and
while said first micro-object and said second micro-object are grouped, combining said first micro-object and said second micro-object in said liquid medium to produce a combined biological micro-object.

2. The process of claim 1, wherein:
said selecting comprises trapping said first micro-object in a first light trap directed into said micro-fluidic device, and trapping said second micro-object in a second light trap directed onto said micro-fluidic device; and
said grouping comprises merging said first light trap and said second light trap and thereby forming a merged light trap trapping said first micro-object and said second micro-object.

3. The process of claim 2, wherein said combining comprises moving said merged light trap through a combining region of said micro-fluidic device.

4. The process of claim 3, wherein said combining region comprises a chemical that facilitates said combining.

5. The process of claim 2, wherein said combining comprises moving said merged light trap directly between a first electrode and a second electrode to which a power source is connected.

6. The process of claim 2, wherein:
said combining comprises moving said merged light trap through a compression passage defined by opposing walls, and
a width of said compression passage is less than a combined width of said first micro-object and said second micro-object.

7. The process of claim 2, wherein:
said selecting comprises generating a first virtual conveyer comprising a plurality of said first light traps for trapping and conveying ones of a plurality of said first micro-objects in said medium, and generating a second virtual conveyer comprising a plurality of said second light traps for trapping and conveying ones of a plurality of said second micro-objects in same medium; and
said paring comprises merging said first light traps with said second light traps to form a third virtual conveyer comprising a plurality of said merged light traps for conveying groups of said first micro-objects and said second micro-objects in said medium.

8. The process of claim 1 further comprising:
creating a first flow of said medium into a chamber, said first flow resulting in a first laminar flow in said chamber,
creating a second flow of said medium into said chamber, said second flow resulting in a second laminar flow in said chamber, and
creating a third flow into said chamber, said third flow resulting in a third laminar flow in said chamber,
wherein:
said selecting comprises selecting said first micro-object from said first laminar flow, and selecting said second micro-object from said second laminar flow; and
said combining comprises moving said grouped first micro-object and second micro-object into said third laminar flow.

9. The process of claim 8, wherein said third flow is of a chemical that facilitates said combining.

10. The process of claim 1, wherein:
said selecting comprises selecting said first micro-object in a flow of said medium in a first channel, and selecting said second micro-object in a flow of said medium in a second channel; and
said grouping comprises moving said first micro-object from said flow of said medium in said first channel to a barrier in a chamber containing said medium, and moving said second micro-object from said flow of said medium in said second channel to said barrier, wherein said chamber is disposed between said first channel and said second channel.

11. The process of claim 10, wherein said combining comprises flowing a chemical that facilitates said combining into said chamber.

12. The process of claim 1 further comprising, while said first micro-object and said second micro-object are grouped but before said combining, breaching a membrane of said first micro-object or a membrane of said second micro-object.

13. The process of claim 12, wherein said breaching comprises:
piercing said membrane of said first micro-object or said membrane of said second micro-object with a sharp, physical structure,
breaching said membrane of said first micro-object or said membrane of said second micro-object with a laser,
applying ultrasonic vibrations to said one of said first micro-object or said second micro-object, or
applying an electrical stimulus to said one of said first micro-object or said second micro-object.

14. The process of claim 1, wherein said selecting and said grouping comprise:
disposing said first micro-object in a first flow of said medium in a first channel connected by a first passage to a third channel, wherein a width of said first passage is less than a size of said first micro-object; and
disposing said second micro-object a second flow of said medium in a second channel connected by a second passage to said third channel, wherein a width of said second passage is less than a size of said second micro-object
wherein:
friction forces stop and hold said first micro-object in said first passage and said second micro-object in said second passage, and
thereafter a combination of said first flow and said second flow overcome said friction forces and push said first micro-object and said second micro-object into said third channel.

15. The process of claim 1 further comprising:
repeating said selecting, said grouping, and said combining to form a plurality of combined micro-objects; and
selecting a subset of less than all of said combined micro-objects based on a predetermined characteristic of said combined micro-objects.

16. The process of claim 1, wherein:
said selecting comprises selecting a third biological micro-object from said plurality of micro-objects in said liquid medium in said micro-fluidic device;
said grouping comprises grouping said third micro-object with said first micro-object and said second micro-object in said liquid medium in said micro-fluidic device; and
said combining comprises, while said first micro-object, said second micro-object, and said third micro-object are grouped, combining said first micro-object, said second micro-object, and said third micro-object in said liquid medium to produce said combined biological micro-object.

17. An apparatus for combining biological micro-objects, said apparatus comprising:
one or more enclosures configured to contain a liquid medium in which are disposed first biological micro-objects and second biological micro-objects;
a grouping mechanism configured to group ones of said first micro-objects with ones of said second micro-object to produce micro-object groups, wherein each said micro-object group comprises one of said first micro-objects and one of said second micro-objects; and
a combining mechanism configured to combine said first micro-objet and said second micro-object in each said micro-object group.

18. The apparatus of claim 17, wherein said grouping mechanism comprises an optoelectronic tweezers (OET) device configured selectively to trap and move selected ones of said first micro-objects and said second micro-objects in said medium.

19. The apparatus of claim 17, wherein:
said enclosures comprise channels for said medium, and
said grouping mechanism comprises:
a first one of said channels having a width that is larger than said first micro-objects,
a second one of said channels having a width that is larger than said second micro-objects,
a third one of said channels,
a first passage from said first one of said channels to said third one of said channels, wherein a width of said first passage is smaller than said first micro-objects, and
a second passage from said second one of said channels to said third one of said channels, wherein a width of said second passage is smaller than said second micro-objects.

20. The apparatus of claim 17, wherein said combining mechanism comprises a region of said enclosure(s) containing a chemical that facilitates said combining of said micro-object groups.

21. The apparatus of claim 17, wherein said combining mechanism comprises:
a first electrode and a second electrode spaced apart from said first electrode, and
a power source connected to said first electrode and said second electrode.

22. The apparatus of claim 17, wherein said combining mechanism comprises a compression passage defined by opposing walls that are spaced apart by a distance that is less than a width of one of said micro-object groups.

23. The apparatus of claim 17, wherein said enclosures comprise:
a first channel for said medium,
a second channel for said medium, and
chambers connected to and disposed between said first channel and said second channel.

24. The apparatus of claim 23, wherein said combining mechanism comprises:
barriers disposed in each of said chambers, each said barrier configured to hold one of said micro-object groups, and
a third channel connected to each of said chambers.

25. The apparatus of claim 17 further comprising a breaching mechanism configured to breach a membrane of one of said first micro-object or said second micro-object in each said micro-object group.

26. The apparatus of claim 17, wherein said breaching mechanism comprises a sharp, physical structure disposed in one of said enclosures, a laser device, an ultrasonic device, or an electrical stimulus device.

27. An apparatus of claim 17, wherein:
said grouping mechanism is further configured to group ones of said first micro-objects with ones of said second micro-objects and ones of third micro-objects in said liquid medium, wherein each said micro-object group comprises one of said first micro-objects, one of said second micro-objects, and one of said third micro-objects; and
said combining mechanism is further configured to combine said first micro-object, said second micro-object, and said third micro-object in each said micro-object group.

28. An apparatus for combining biological micro-objects, said apparatus comprising:
grouping means for grouping one of a plurality of first biological micro-objects with one of a plurality of second biological micro-object in a liquid medium; and
combining means for combining said one of said first micro-objects and one of said second micro-objects into a combined biological micro-object.

29. The apparatus of claim 28, wherein said grouping means comprises means for generating dielectrophoresis (DEP) forces to select and move said one of said first micro-objects and said one of said second micro-objects in said medium.

30. The apparatus of claim 28 further comprising breaching means for breaching a membrane of said one of said first micro-object or a membrane of said one of said second micro-objects;

31. The apparatus of claim 30, wherein said breaching means comprises means for piercing said membrane of said one of said first micro-objects or said membrane of said one of said second micro-objects.

32. The apparatus of claim 28, wherein said combining means comprises means for exposing said one of said first micro-objects and said one of said second micro-objects to a chemical that effects said combining.

33. The apparatus of claim 28, wherein said combining means comprises means for generating an electric field of sufficient magnitude to effect said combining.

34. The apparatus of claim 28, wherein said combining means comprises means for applying sufficient pressure to said one of said first micro-objects and said one of said second micro-objects to effect said combining.

35. An apparatus of claim 28, wherein:
said grouping means is further for grouping said one of said plurality of first biological micro-objects with said one of said plurality of second biological micro-object with one of a plurality of third biological micro-objects in said liquid medium; and
said combining means is further for combining said one of said first micro-objects, said one of said second micro-objects, and said one of said third micro-objects into a combined biological micro-object.
